(19) United States
(12) Patent Application Publication
(10) Pub. No.: US 2011/0306042 A1
(43) Pub. Date: Dec. 15, 2011

(54) DETERMINATION OF CHROMATIN CONFORMATION

(75) Inventor: Yann Jouvenot, Benicia, CA (US)

(73) Assignee: Bio-Rad Laboratories, Inc., Hercules, CA (US)

(21) Appl. No.: 13/152,012

(22) Filed: Jun. 2, 2011

Related U.S. Application Data
(60) Provisional application No. 61/351,569, filed on Jun. 4, 2010.

(51) Int. Cl. C12Q 1/68; B82Y 15/00

(52) U.S. Cl. 435/6.1; 977/781; 977/902

(57) ABSTRACT

Methods and kits for determining histone modification or epigenetic status are provided.

### CROSS-REFERENCE TO RELATED PATENT APPLICATIONS

The present application claims benefit of priority to U.S. Provisional Patent Application No. 61/351,569, filed Jun. 4, 2010, which is incorporated by reference for all purposes.

### BACKGROUND OF THE INVENTION

Within the eukaryotic cell nucleus, genetic information is organized in a highly conserved structural polymer, termed chromatin, which supports and controls the crucial functions of the genome. The chromatin template undergoes dynamic changes during many genetic processes. These include necessary structural reorganizations that occur during DNA replication and cell cycle progression, spatially and temporally coordinated gene expression, as well as DNA repair and recombination events. The fundamental repeating unit of chromatin is the nucleosome, which consists of 146 base pairs of DNA wrapped around an octamer of core histone proteins, H2A, H₂B, H3, and H4. Linker histones of the H1 class associate with DNA between single nucleosomes, establishing a higher level of organization, the so-called ‘solenoid’ helical or zig-zag fibers (30 nm fibers). Chromatin architecture beyond the 30 nm fibers is less clear, but folding and unfolding of putative superstructures is thought to have a pronounced impact on genomic function and gene activity.

Core histone proteins are evolutionary conserved and consist mainly of flexible N-terminal tails protruding outward from the nucleosome, and globular C-terminal domains making up the nucleosome scaffold. Histones function as acceptors for a variety of post-translational modifications, including acetylation, methylation and ubiquitination of lysine (K) residues, phosphorylation of serine (S) and threonine (T) residues, methylation of arginine (R) residues as well as sumoylation, biotinylation, and deimination. The different histone modifications and the corresponding enzymatic systems that maintain them have been reviewed extensively in the recent literature (e.g. Zhang et al., “Transcription Regulation by Histone Methylation: Interplay Between Different Covalent Modifications of the Core Histone Tails,” Genes Dev 15:2343-2360 (2000); Kouzarides, “Histone Methylation in Transcriptional Control,” Curr Opin Genet Dev 12:198-209 (2002); Lachner et al., “The Many Faces of Histone Lysine Methylation,” Curr Opin Cell Biol 14:286-298 (2002); Berger, Histone Modifications in Transcriptional Regulation,” Curr Opin Genet Dev 12:142-148 (2002); and Eberharter et al., “Histone Acetylation: A Switch Between Repressive and Permissive Chromatin,” Second in Review Series on Chromatin Dynamics. EMBO Rep 3:224-229 (2002)). Combinations of post-translational marks on single histones, single nucleosomes and nucleosomal domains establish local and global patterns of chromatin modification that may specify unique downstream functions (Strahl et al., “The Language of Covalent Histone Modifications,” Nature 403:41-45 (2000); Turner, “Histone Acetylation and an Epigenetic Code,” Bioessays 22:836-845 (2000)). These patterns can be altered by multiple extracellular and intracellular stimuli, and chromatin itself has been proposed to serve as signaling platform and to function as a genomic integrator of various signaling pathways (Cheung et al., “Signaling to Chromatin Through Histone Modifications,” Cell 103:263-271 (2000)).

DNA from eukaryotic cells, including cancer cells, often displays somatic changes in DNA methylation. See, e.g., E. R. Fearon, et al, Cell 61:759 (1990); P. A. Jones, et al., Cancer Res. 46:461 (1986); R. Holliday, Science 238:163 (1987); A. De Bustros, et al., Proc. Natl. Acad. Sci. USA 85:5693 (1988); P. A. Jones, et al., Adv. Cancer Res. 54:1 (1990); S. B. Baylin, et al., Cancer Cells 3:383 (1991); M. Makos, et al., Proc. Natl. Acad. Sci. USA 89:1929 (1992); N. Ohtani-Fujita, et al., Oncogene 8:1063 (1993).

DNA methylases transfer methyl groups from the universal methyl donor S-adenosyl methionine to specific sites on the DNA. Several biological functions have been attributed to the methylated bases in DNA. The most established biological function is the protection of the DNA from digestion by cognate restriction enzymes. This restriction modification phenomenon has, so far, been observed only in bacteria.

Mammalian cells, however, possess different methylases that exclusively methylate cytosine residues on the DNA that are 5′ neighbors of guanine (CpG). This methylation has been shown by several lines of evidence to play a role in gene activity, cell differentiation, tumorigenesis, X-chromosome inactivation, genomic imprinting and other major biological processes (Razin, A., H., and Riggs, R. D. eds. in DNA Methylation Biochemistry and Biological Significance, Springer-Verlag, N.Y., 1984).

### BRIEF SUMMARY OF THE INVENTION

The present invention provides methods of analyzing chromatin structure and/or epigenetic status in chromosomal DNA of a cell. In some embodiments, the methods comprise:

(i.) contacting a first DNA endonuclease to nuclear DNA in the cell, thereby introducing single-stranded nicks in the nuclear DNA;
(ii.) labeling the nicks in the DNA with a first detectable label to generate labeled nicks;
(iii.) ligating the labeled nicks in the DNA;
(iv.) after step (i.), (ii.), or (iii.), purifying the DNA from the cell to generate purified genomic DNA;
(v.) after steps (i.)-(iv.), contacting a second DNA endonuclease to the purified genomic DNA to introduce single stranded nicks in the purified genomic DNA, wherein the second DNA endonuclease is different than the first endonuclease;
(vi.) labeling the nicks introduced by the second endonuclease in the purified genomic DNA with a second detectable label that is distinguishable from the first detectable label.

In some embodiments, the methods further comprise: (vii.) determining the identity of a region of interest in the genomic DNA based on the number and/or pattern of the second detectable labels in the genomic DNA and determining the presence, absence, or quantity of the first detectable labels in the region of interest, wherein the presence or quantity of the first detectable labels corresponds to chromatin structure and/or epigenetic status in the DNA region of interest in the nucleus of the cell.

In some embodiments, the method further comprises, between steps (vi.) and (vii.), ligating the labeled nicks in the purified genomic DNA.

In some embodiments, steps (ii.)-(iii.) occur before step (iv.).

In some embodiments, steps (ii.)-(iii.) occur after step (iv.).

In some embodiments, prior to step (i) a cell membrane and nucleus of the cell is permeabilized to allow for entry of the first endonuclease into the nucleus.

In some embodiments, the first endonuclease is a DNase. In some embodiments, the DNAse is DNase I.

In some embodiments, the second endonuclease is a sequence-specific single-stranded restriction endonuclease. In some embodiments, the restriction endonuclease recognizes a 4, 5, 6, 7, or 8 base pair recognition sequence.

In some embodiments, the first and second detectable labels are fluorescent labels that produce signal at different wavelengths from each other.

In some embodiments, step (vii) comprises: linearizing DNA molecules generated in step (vi) and recording the position of the first and/or second labels on the DNA molecules; and correlating the position of the second labels on the DNA molecules to a region of interest in the genomic DNA. In some embodiments, the linearizing step comprises passing single DNA molecules generated in step (vi) through a nanochannel. In some embodiments, the passing step is performed in parallel with multiple single DNA molecules in a plurality of nanochannels.

In some embodiments, the accessibility of the first enzyme to the nuclear DNA is correlated with chromatin state (e.g., quantity of modification).

In some embodiments, the first enzyme is linked to an affinity agent having affinity for a histone modification or cytosine methylation such that the accessibility of the first enzyme to the nuclear DNA is correlated with lack of histone modification status or methylation status. In some embodiments, the affinity agent is an antibody. In some embodiments, the affinity agent has affinity for a histone modification. In some embodiments, the affinity agent has affinity for methylated DNA. In some embodiments, the affinity agent is Methyl CpG binding protein (MBP). In some embodiments, the histone modification is selected from histone methylation, histone acetylation, histone ubiquitinylation, histone phosphorylation, histone sumoylation, histone biotinylation, histone deimination, DNA (e.g., adenosine or cytosine) methylation, or other DNA or histone modification.

In some embodiments, the method further comprises, after step (vii) and before step (viii): contacting a third DNA endonuclease to the purified genomic DNA to introduce single stranded nicks in the purified genomic DNA, wherein the third DNA endonuclease recognizes a different DNA recognition that the first and second DNA endonucleases; and labeling the nicks in the purified genomic DNA with a third detectable label that is distinguishable from the first and second detectable labels.

In some embodiments, the method is performed on a single cell.

In some embodiments, the method is performed on a plurality of cells.

Kits (e.g., for performing the methods described herein) are provided. In some embodiments, the kit comprises a first DNA endonuclease; a second DNA endonuclease different from the first DNA endonuclease; a first detectable label; a second detectable label different from the first detectable label; a DNA polymerase; and a ligase. In some embodiments, the first endonuclease is fused to a protein domain having affinity to a particular histone modification or cytosine methylation. In some embodiments, the first endonuclease is a DNase.

In some embodiments, the second endonuclease is a sequence-specific restriction endonuclease. In some embodiments, the first and second labels are fluorescent labels. In some embodiments, the labels are linked to deoxynucleotide triphosphates. In some embodiments, the kit further comprises a cell permeabilizing agent and/or formalin or formaldehyde or other cell-fixing agent.

Other aspects of the invention will be clear from reading the remainder of this document.

### DEFINITIONS

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but which functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Accessibility” of a DNA region to a polypeptide, as used herein, refers to the ability of a particular DNA region in a chromosome of a cell to be contacted and nicked by a particular polypeptide. Without intending to limit the scope of the invention, it is believed that the particular chromatin structure comprising the DNA region will affect the ability of a polypeptide to nick the particular DNA region. For example, the DNA region may be wrapped around histone proteins and further may have additional nucleosomal structure that prevents, or reduces access of, the polypeptide to the DNA region of interest.

“Epigenetic status” refers to the presence or absence or amount of epigenetic modification (such as DNA methylation or histone modifications) in a particular DNA region of interest.

“Chromatin structure” refers to the level of compaction and/or accessibility of the chromatin in the nucleus. This is influenced by the presence, absence, or quantity of epigenetic marks in the DNA (DNA methylation, histone modifications). The structure of the chromatin has a direct effect on the level of expression of genes, and influences other nuclear processes (e.g. replication, topological localization).

“Chromosomal DNA” refers to DNA that is present in or purified from a chromosome.

“Nuclear DNA” refers to DNA present in a nucleus of a cell, ideally that maintains the chromatin structure found within the cell.

“Labeled nicks” refer to single-stranded nicks in DNA that have a label attached such that the location of the nicks can be determined based on the presence and location of the label.

“Single-stranded restriction endonuclease” refers to an enzyme that introduces single-stranded nicks in DNA based on a specific recognition sequence in the DNA. The recognition sequence will be have at least 4 nucleotides and can have, for example, 4, 5, 6, 7, 8, 9, 10, or more contiguous nucleotides. Single-stranded restriction enzymes do not cleave DNA at both strands upon biding or recognizing it cognate recognition sequence. A “Recognition sequence” of a restriction enzyme refers to a sequence, typically contiguous but sometimes in two separate parts, that is recognized by a restriction enzyme prior to nicking of cleavage (depending on the type of enzyme). Some single-stranded restriction enzymes introduce nicks within the recognition sequence, while others introduce nicks near, but not within the recognition sequence.

An “affinity agent” refers to a protein or non-protein molecule that has specificity for a particular target molecule.

The phrase “specificity for” a particular target molecule refers to a binding reaction, or capability of having a binding reaction when the target is present) that is determinative of the presence of the target in a heterogeneous population of proteins and other biologics. Thus, the specified affinity agent binds to a particular protein at least two times the background and do not substantially bind in a significant amount to other proteins present in the sample. Typically a specific or selective reaction will be at least twice background signal or noise and more typically more than 10 to 100 times background.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Naturally occurring immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively. The locations of CDR and FR regions and a numbering system have been defined by, e.g., Kabat et al. (Kabat et al., Sequences of Proteins of Immunological Interest, Fifth Edition, U.S. Department of Health and Human Services, U.S. Government Printing Office (1991)).

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see FUNDAMENTAL IMMUNOLOGY (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

### DETAILED DESCRIPTION

### I. Introduction

Methods of analyzing chromatin structure and/or epigenetic status of genomic DNA are provided. The methods involve introducing labels in nuclear DNA in a cell in a manner that is dependent upon the chromatin structure and/or epigenetic status of regions in the nuclear DNA. Generally, a first nicking polypeptide having single-stranded nicking activity is contacted to the nuclear DNA in the cell such that the nicking polypeptide nicks different regions of the DNA with different frequency depending on the chromatin structure and/or epigenetic status of the region. More details of how this can be achieved are provided below, but one example is use of a nicking polypeptide that nicks DNA with more frequency in DNA having “loose” chromatin structure (e.g., euchromatin) than in “tighter” chromatin (e.g., heterochromatin). In another example, the nicking polypeptide is linked to an affinity agent such that the nicking polypeptide is targeted to a particular chromatin or epigenetic structure (e.g., histone modification, cytosine or other DNA methylation) and thus nicks DNA in regions with more of those structures more than in regions compared to other regions.

Once nicks are introduced into the nuclear DNA, the nicks are subsequently labeled with a first label and blocked from subsequent labeling by a second label (discussed further below), for example by ligating the nicks back together, thereby repairing the nick. The DNA is subsequently nicked by a second nicking polypeptide in a sequence-specific manner, for example using a single-stranded nicking enzymes with an affinity for particular recognition sequences, or other manner that reproducibly occurs in the genome. These secondary nicks are then labeled with a second label.

By determining the quantity and/or pattern of second labels in the DNA, the identity of one or more region of interest can be determined. The presence, absence or quantity of the first label in the one or more region of the DNA can then be correlated to the presence, absence or quantity of chromatin structure and/or epigenetic status based on the sensitivity of the first nicking polypeptide.

The methods of the invention are useful, for example, for any diagnostic, prognostic or other personalized medicine application where chromatin structure and/or epigenetic status of one or more DNA region is or may be correlated with a particular disease or condition.

### II. Generation of single-stranded nicks

As noted above, the methods provide for at least two different nicking steps. A first nicking step is sensitive or dependent upon a particular chromatin structure and/or epigenetic status. The first nicking can be achieved using any nicking polypeptide (i.e., a polypeptide having single-stranded nicking activity) whose activity varies depending on chromatin structure and/or epigenetic status.

Single-stranded nicking refers to an enzymatic activity by which a polypeptide causes a breakage in one of the two strands of a DNA molecule. While it will be appreciated that a nicking enzyme may be active more than once on a particular molecule and thus may by chance introduce nicks on both strands at different times, nicking activity can be distinguished from, for example, double-stranded endonuclease activity, which as a primary activity cleave both strands of the DNA backbone.

In some embodiments, the nicking polypeptide has an activity that is chromatin structure and/or epigenetic status sensitive. In some embodiments, the enzyme has sufficient size that sufficient steric hindrance in some chromatin regions result in a measurable differential activity, thereby generating measurably more nicks in some regions of chromatin than others. Without intending to limit the invention, it is believed that the differences in accessibility reflect differences in “open” and “closed” chromatin, i.e., heterochromatin compared to euchromatin. An example of such an enzyme includes, including but not limited to, DNase I or a micrococcal nuclease. DNase I has been shown previously to have differential activity between loose and tight chromatin. See, e.g., Rando & Chang, Annu. Rev. Biochem 2009; 78, 245-271. However, it is believed that other types of enzymes with single-stranded nicking activity, including for example sequence-specific single-stranded restriction enzymes will also have chromatin-type specific activity due to steric hindrance issues. Moreover, in some embodiments, an enzyme or fragment thereof having single-stranded nicking activity is fused to one or more other protein sequence or domain fusion partner to generate a polypeptide that differentially nicks different chromatin based on chromatin structure due to steric hindrance created at least in part by the fusion partner.

In some embodiments, the nicking polypeptide has an activity that is chromatin structure and/or epigenetic status dependent, i.e., is preferentially active where a certain structure or epigenetic modification is present compared to absent (or for example in high density compared to low density). For example, in some embodiments, the nicking polypeptides specifically bind or otherwise recognize histone methylation, histone acetylation, histone ubiquitinylation, DNA (e.g., adenosine or cytosine) methylation, or other DNA or histone modification. In some embodiments, the nicking polymerase is engineered for such specificity by fusing at least a protein fragment having nicking activity to an affinity agent with specificity for a particular histone or epigenetic modification such as those listed above. Engineering specificity of restriction enzymes (albeit in double-stranded restriction endonucleases) has been described, for example, in Chan et al., Nuc. Acids. Res. 35(18):6238-6248 (2007).

Any sequence non-specific endonuclease having nicking activity (e.g., DNase I) or sequence-specific endonuclease having nicking activity can be used according to the present invention. DNases used can include naturally occurring DNases as well as modified DNases. Exemplary DNases, include but are not limited, to Bovine Pancreatic DNase I (available from, e.g., New England Biolabs.

A number of sequence-specific endonucleases having nicking activity have been described and can be used in the methods described herein. Nt.BstNBI is a naturally occurring thermostable nicking endonuclease cloned from Bacillus Stereothermophilus. Nb.BsrDI and Nb.BtsI are naturally occurring large subunits of thermostable heterodimeric enzymes. Nt.AlwI, a derivative of the restriction enzyme AlwI, has been engineered to behave in the same way. Both nick just outside their recognition sequences. Nb.BbvCI and Nt.BbvCI are alternative derivatives of the -heterodi-meric restriction enzyme BbvCI, each engineered to possess only one functioning catalytic site. These two enzymes nick within the recognition sequence but on opposite strands. Nb.BsmI is a bottom-strand specific variant of BsmI discovered from a library of random mutants. Other nicking enzymes as well as methods of generating single-stranded nicking enzymes by modifying double-stranded endonucleases are described in US Patent Publication No. 2008/0213860.

As noted above, in some embodiments the nicking polypeptide comprises, or is linked to, an affinity agent having affinity for a chemical moiety or structure associated with a particular epigenetic status of chromatin structure. For example, the affinity agent can have binding specificity to one of histone methylation, histone acetylation, histone ubiquitinylation, histone phosphorylation, histone sumoylation, histone biotinylation, histone deimination, DNA (e.g., adenosine or cytosine) methylation, or other DNA or histone modification. In some embodiments, the affinity agent is an antibody having specificity for one of the above-described modifications. For example, methyl-cytosine binding antibodies have been described. Antibodies specific for DNA methylation and histone modification are commercially available, e.g., from AbCam (Cambridge, Mass.). Alternatively, non-antibody proteins can be generated for binding such modifications. A number of technologies for generating such non-antibody binding proteins are known including but not limited to adnectins and avimers. Nucleic acid-based aptamers and peptides, for example, can also be screened and developed as affinity agents.

Exemplary methyl-cytosine binding proteins include, e.g., MeCP2 (methyl-CpG binding protein 2), MBD1, MBD2, MBD3, MBD4, MBD2-CTH1, and MDB2-CTH2. See, e.g., US Patent Publication Nos. 20060099580; 20100112585; and U.S. Pat. No. 7,425,415.

As noted above, the methods involve at least two separate nicking steps: a first nicking step that occurs on native chromatin in a cell nucleus and a second nicking step that occurs on purified DNA and is not chromatin or epigenetic status-specific. The first nicking step occurs in a cell and thus involves delivery of the nicking polypeptide into the cell of interest. In some embodiments, therefore, the cell or cells of interest are permeabilized to allow for delivery of the nicking polypeptide through the cell membrane and into the nucleus.

Cell membranes can be permeabilized or disrupted in any way known in the art. The methods involve contacting the genomic DNA prior to isolation of the DNA and thus methods of permeabilizing or disrupting the cell membrane will not disrupt the structure of the genomic DNA of the cell such that nucleosomal or chromatin structure is destroyed.

In some embodiments, the cell membrane is contacted with an agent that permeabilizes or disrupts the cell membrane. Lysolipids are an exemplary class of agents that permeabilize cell membranes. Exemplary lysolipids include, but are not limited to, lysophosphatidylcholine (also known in the art as lysolecithin) or monopalmitoylphosphatidylcholine. A variety of lysolipids are also described in, e.g., WO/2003/052095.

Non ionic detergents are an exemplary class of agents that disrupt cell membranes. Exemplary nonionic detergents, include but are not limited to, NP40, Tween 20 and Triton X-100.

In some embodiments, the nicking polypeptide and the permeabilization agent are simultaneously delivered to the cell. Thus, in some embodiments, a buffer comprising both agents is contacted to the cell. The buffer should be adapted for maintaining activity of both agents (permeabilization agent and nicking polypeptide) while maintaining the structure of the cellular chromatin.

Alternatively, electroporation or biolistic methods can be used to permeabilize a cell membrane such that a nicking polypeptide is introduced into the cell and can thus contact the genomic DNA. A wide variety of electroporation methods are well known and can be adapted for delivery of DNA modifying agents as described herein. Exemplary electroporation methods include, but are not limited to, those described in WO/2000/062855. Biolistic methods include but are not limited to those described in U.S. Pat. No. 5,179,022.

In some embodiments, the cells will be chemically cross-linked or “fixed”. For example, the cell or cells can be treated with formalin or formaldehyde as is known in the art to fix the cells prior to permeabilization.

The amount and concentration of nicking enzyme used will depend on the activity of the particular nicking polypeptide used. Generally, a sufficient amount and concentration will be used to produce a detectable amount of nicking in the genomic DNA such that one can detect a difference in nicking in different genomic regions.

The later nicking step occurs once the genomic DNA has been purified from the cell(s). Once the DNA is purified, the DNA no longer includes chromatin structures. The DNA is nicked in the later nicking step under conditions, and with nicking polypeptides, whose activity are not dependent upon chromatin or epigenetic status. Generally, the later nicking step will involve a sequence-specific nicking endonuclease or other enzyme having a known or reproducible nicking pattern in the genomic DNA such that the placement of the nicks by the nicking polypeptide used in this step can be later correlated with a particular region of DNA.

In some embodiments, more than one nicking enzyme step and subsequent labeling step are performed once the genomic DNA is purified. Because a main function of the post-purification nicking steps is to provide a recognizable, reproducible nicking pattern that can be used to determine a particular DNA region of interest based on the signature nicking pattern, it can in some cases be helpful to use two or more different nicking/labeling rounds so that regions with relatively few recognition sequences for one nicking enzyme will be nicked with increased frequency by another nicking enzyme, thereby allowing for improved “viewing” of the entire genome.

Any type of eukaryotic cells can be used in performance of the methods. Exemplary cells include, e.g. animal, plant or fungal cells. Exemplary animal cells include but are not limited to mammalian cells, e.g., human, primate, mouse, rat, bovine, canine or other mammalian cells. Cells used can be from any cell or tissue type. Indeed, in some embodiments, it will be useful to compare chromatin modification and epigenetic status between different cell types or between the same cell types between individuals.

The methods can be performed on single cells or combinations of more than one cell. In addition, a plurality of single cells or mixtures can be processed in parallel as desired.

### III. Labeling nicks

Following any of the nicking steps described above, the nicks are labeled so that the presence and location of the nicks can be determined. In some embodiments, a detectable label is introduced at the site of the nick. In some embodiments, the nicks can be labeled by extending a 3′ end of a nicked strand to add one to more labeled nucleotides. This can be achieved, for example, by contacting the nicks with at least one labeled nucleotide triphosphate (e.g., labeled dATP, dTTP, dCTP, dUTP, and/or dGTP or analogs thereof) and a DNA polymerase under conditions in which the polymerase extends the nick and introduces at least one labeled nucleotide. Exemplary polymerases include those with 5′-3′ exonuclease activity, including but are not limited to, E. coli DNA polymerase.

The label may function to: (i) provide a detectable signal; (ii) interact with a second label to modify the detectable signal provided by the first or second label, e.g. to give FRET (fluorescence resonance energy transfer); (iii) stabilize interactions or increase affinity of binding, with antigen or ligand; (iv) affect mobility, e.g. electrophoretic mobility or cell-permeability, by charge, hydrophobicity, shape, or other physical parameters, or (v) provide a capture moiety, to modulate ligand affinity, antibody/antigen binding, or forming ionic complexes.

Numerous labels are available which can be generally grouped into the following categories:

Radioisotopes (radionuclides), such as 3H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. Radioisotope labelled antibodies are useful, for example, in targeted imaging. The antibody can be labeled with ligand reagents that bind, chelate or otherwise complex a radioisotope metal. See, e.g., Current Protocols in Immunology, Volumes 1 and 2, Coligen et al, Ed. Wiley-Interscience, New York, N.Y., Pubs. (1991). Chelating ligands which may complex a metal ion include DOTA, DOTP, DOTMA, DTPA and TETA (Macrocyclics, Dallas, Tex.).

Additional labels include, e.g., fluorescent labels such as rare earth chelates (europium chelates), fluorescein types including FITC, 5-carboxyfluorescein, 6-carboxy fluorescein; rhodamine types including TAMRA; dansyl; Lissamine; cyanines; phycoerythrins; Texas Red; and analogs thereof. Fluorescent dyes and fluorescent label reagents include those which are commercially available from Invitrogen/Molecular Probes (Eugene, Oreg.) and Pierce Biotechnology, Inc. (Rockford, Ill.).

Labeling can be achieved either following purification of the DNA or, if desired, in the cell (in some embodiments, following the first nicking step). When a DNA purification scheme that substantially maintains the intact nature of nicked DNA is employed, one can label nicks from the first nuclear nicking reaction following DNA purification. In embodiments in which the nicked DNA has been purified, nicked DNA is labeled with a polymerase, standard conditions for extension reactions for the polymerase employed can be used so long as excessive DNA shearing is avoided. In embodiments in which the nicked DNA is still present in a cell (following the first nicking reaction), the labeling reaction can be performed by delivering the polymerase and labeled nucleotides to the DNA in the cell (e.g., by similar methods as delivery of the nicking polypeptide) and submitting the mixture to conditions to allow for labeling to occur.

The labeling steps will generally use a label that is distinguishable from other labels in the DNA. Thus, for example, the label used to label the nicks created in the first nicking step will be distinguishable from the label used to label the nicks created in the second or subsequent nicking steps. Similarly, if there is a third nicking step, the labels used to label the nicks from the third nicking step will be distinguishable from the labels used in the first two nicking steps. When fluorescent labels are used, the labels can be distinguishable, for example, by using labels whose fluorescent signal is at a different wave length than other labels.

### IV. Ligating Nicks

Labeled nicks can be ligated following the labeling reaction. Generally, in situations where a subsequent nicking step will occur (e.g., after the first nicking reaction, and if there is a third nicking reaction then after the second nicking reaction), the earlier nicks are labeled and then ligated. By ligating the nicks, the nicks are not available for further labeling and thus when a subsequent step introduces new nicks, the new nicks can be labeled without labeling a significant, if any, number of nicks from previous nicking steps. This allows for at least two nicking reactions that nick under different criteria to be labeled differently thereby making the two types of labels distinguishable.

Ligation, when performed on purified DNA, can be performed under standard conditions for the ligase such that substantially all of the nicks in the DNA are ligated, thereby reestablishing the intact double strand, albeit with one or more additional nucleotides introduced from the labeling reaction.

In some embodiments, following the first nicking reaction, the labeling and in some cases, ligation steps are performed in the cell prior to purifying the DNA. In those cases, the ligase is delivered to the nuclear DNA by similar methods as delivery of the nicking polypeptide. Alternatively, the ligation can be performed on purified DNA.

In some embodiments, following the final nicking step, the DNA is not ligated. Nevertheless, in some cases, following the final nicking step, the DNA is ligated, for example to ensure that the integrity of the DNA remains intact.

### V. DNA Purification

As noted above, DNA purification can occur after the first nicking step, after the first labeling step or after the first ligating step, but in any case occurs before the nicking step (i.e., typically the second nicking step) that is performed under conditions that are independent of chromatin or epigenetic status.

In some embodiments, following the DNA modification/cleavage step, genomic DNA is isolated from the cells according to any method available. Essentially any DNA purification procedure can be used so long as it results in DNA of acceptable purity and without significant shearing for the subsequent detection step(s). For example, standard cell lysis reagents can be used to lyse cells. Optionally a protease (including but not limited to proteinase K) can be used. DNA can be isolated from the mixture as is known in the art. In some embodiments, phenol/chloroform extractions are used and the DNA can be subsequently precipitated (e.g., by ethanol) and purified. In some embodiments, RNA is removed or degraded (e.g., with an RNase), if desired.

Any method of DNA purification can be used that maintains the DNA substantially intact, i.e., at least such that most fragments are greater than 20, 40, 60, 80, 100 kb or more. Thus, it can be desirable to purify and manipulate the subsequent purified DNA in soft agarose or similar material to protect from extensive shearing. Similarly, it can be helpful to generally treat samples as one would for samples to be examined using Pulse Field Gel Electrophoresis or other large DNA molecule analyses such that the DNA strands do not experience substantial shearing. The amount of shearing tolerated by the method will depend on the precise method for ultimately detecting the labels in the DNA.

### VI. Determining the Identity of DNA Region of Interest

Following chromatin or epigenetic-specific labeling and non-specific labeling of the DNA, the chromatin or epigenetic structure of a DNA region can be inferred based on the presence, absence or quantity of label associated with the chromatin or epigenetic-specific nicking step. For example, where a nicking enzyme accesses (and therefore nicks) euchromatic regions of the genome, but does not access heterochromatic regions, the absence or relative in frequency of nicking in a particular region indicates that the region is heterochromatic. In contrast, if the region contains many labels associated with the initial nicking, then the region can be inferred to be euchromatic. Similarly, where the first nicking polypeptide is targeted (e.g., via an affinity agent) to a particular chromatin or epigenetic structure, then the presence or relative quantity of the label associated with the first nicks indicates the region has a relative abundance of the particular chromatin or epigenetic structure.

Nevertheless, the identity of a particular genomic region will not be evident based on the pattern or relative abundance of the label associated with the first nicking step because the first nicking step is determined by particular chromatin or epigenetic structure. Therefore, to identify a particular region of interest (i.e., to determine a particular genomic sequence or location of a chromosome of a region), one can analyze the abundance and pattern of the label(s) associate to the one or more sequence-specific nicking steps that were performed on the purified DNA. Because these nicking steps are sequence-specific, they occur in a particular abundance and relatively unique pattern that one can then identify in a genomic sequence. Thus, as a simplified example, a particular pattern of a label at the intervals 1 kb, 500 bp, 1.3 kb, 600 bp could be located to particular region of a chromosome because the genomic sequence of the organism is known and there is a unique place at which the above-ordered pattern of restriction sites recognized by the nicking enzyme occurs. Locating or mapping the pattern to a particular region in the genome can be achieved, for example, by computer analysis of possible nicking patterns predicted by nucleotide sequence of the genome and or by computer-based comparison of known nicking patterns to those determined from a sample.

In some embodiments, a select number of DNA regions are analyzed by this method. Alternatively, a genome-wide map of chromatin modifications and/or epigenetic modifications can be created. Without intending to limit the invention to a particular use, it is believed that a select number of regions will be examined in situations where modification of the regions are known to have a particular association, e.g., with a disease or cell phenotype, whereas a genome-wide assessment will be made where it is desired to identify regions of interest that differ between two treatments, cell types, phenotypes, diseases, etc.

Any method of analyzing DNA can be used that allows for the determination of the particular order and pattern of labels in stretches of DNA. In some embodiments, following the final labeling step, the DNA is analyzed by linerarizing single DNA molecules and recording the position of the first and/or second labels (and optionally third or subsequent labels) on the DNA molecules. Linearization can occur, for example, by passing single DNA molecules through a nanochannel or nanochannel array. This type of analysis of DNA is generally described in, e.g., U.S. Pat. Nos. 5,867,266; 7,670,770; 6,790,671; PCT Publication No. WO 00/09757, and US Patent Publication Nos. 2008242556 and 20060084078. For example, in some embodiments, one or more nanochannels are provided and the DNA are transported through the channels while being monitored for signal (e.g., fluorescence) from the labels. In some embodiments, nanofluidics and multicolor fluorescence microscopy are employed to map the DNA, for example as described in Cipriany, et al., Anal. Chem. 82 (6): 2480-2487 (2010). A variety of nano-scale methods of manipulating and detecting DNA are reviewed in, e.g., Levy and Craighead, Chem. Soc. Rev., 2010, 39, 1133-1152. Optical mapping technology is available commercially from, e.g., OpGen, (Gaithersburg, Md.), for example OpGen's nanochannels and/or optical acquisition system can be used to determine a DNA region of interest. Examples of such optical DNA mapping technology are described in, e.g., Shukla, et al., J. Bacteriol. 191(18): 5717-5723 (2009) and Latreille et al., BMC Genomics 8: 321 (2007). Optionally DNA linearization and mapping systems can include, for example, a data processor and data storage units to store or analyze the signal as data received.

Quantification of labels associated with the first nicking step in one or more DNA region can be further improved, in some embodiments, by determining the relative amount (e.g., a normalized value such as a ratio or percentage) of first nicking labels in the DNA region compared to an internal control from that same region. In some embodiments, when comparing between two or more DNA regions, the relative amount of label from the first nicking reaction can be normalized to the amont of label from the second labeling reaction, thereby allowing for efficient comparison between samples.

In some embodiments, the actual or relative amount of label in a region is compared to a control value. Control values can be conveniently used, for example, where one wants to know whether the protein accessibility, chromatin modification or epigenetic status of a particular DNA region exceeds or is under a particular value, or within a particular range. For example, in the situation where a particular DNA region is typically accessible in normal cells, but is inaccessible in diseased cells (or vice versa), one may simply compare the actual or relative quantity of label associated with the first nicking to a control value. Alternatively, a control value can represent past or expected data regarding a control DNA region. In these cases, the actual or relative amount of a control DNA region are determined (optionally for a number of times) and the resulting data is used to generate a control value that can be compared with actual or relative quantity of label associated with the first nicking determined for a DNA region of interest.

The calculations for the methods described herein can involve computer-based calculations and tools. The tools are advantageously provided in the form of computer programs that are executable by a general purpose computer system (referred to herein as a “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### VII. Diagnosis

A DNA region is a target sequence of any length of interest within genomic DNA. Any DNA sequence in geliomic DNA of a cell can be evaluated by the methods described herein. DNA regions can be screened to identify a DNA region of interest that displays different accessibility, chromatin modification, or epigenetic status in different cell types, between untreated cells and cells exposed to a drug, chemical or environmental stimulus, or between normal and diseased tissue, for example. In some embodiments, the methods of the invention are used to identify a DNA region whose change in accessibility, chromatin modification or epigenetic status acts as a marker for disease (or lack thereof) or indicates a particular cell status (e.g., for sorting stem cells and or progenitor cells). Exemplary diseases include but are not limited to cancer. A number of genes have been described that have altered DNA methylation and/or chromatin structure in cancer cells compared to non-cancer cells.

In some embodiments, the DNA region is known to be differentially accessible or have certain chromatin modification or epigenetic status depending on the disease or developmental state of a particular cell. In these embodiments, the methods of the present invention can be used as a diagnostic or prognostic tool. Once a diagnosis or prognosis is established using the methods of the invention, a regimen of treatment can be established or an existing regimen of treatment can be altered in view of the diagnosis or prognosis. For instance, detection of a cancer cell according to the methods of the invention can lead to the administration of chemotherapeutic agents and/or radiation to an individual from whom the cancer cell was detected.

A variety of DNA regions can be detected either for research purposes and/or as a control DNA region to confirm that the reagents were performing as expected. For example, in some embodiments, a DNA region is assayed that is accessible in essentially all cells of an animal or other organism. Such DNA regions are useful, for example, as positive controls for accessibility. In some embodiments, such DNA regions can be found, for example, within or adjacent to genes that are constitutive or nearly constitutive. Such genes include those generally referred to as “housekeeping” genes, i.e., genes whose expression are required to maintain basic cellular function. Examples of such genes include, but are not limited to glyceraldehyde-3-phosphate dehydrogenase (GAPDH) and beta actin (ACTB). DNA regions can include all or a portion of such genes, optionally including at least a portion of the promoter.

In some embodiments, a DNA region comprises at least a portion of DNA that is inaccessible in most cells of an animal. Such DNA regions are useful, for example, as negative controls for accessibility. “Inaccessible” in this context refers to DNA regions whose copies are modified in no more than around 20% of the copies of the DNA region. Examples of such gene sequences include those generally recognized as “heterochromatic” and include genes that are only expressed in very specific cell types (e.g., expressed in a tissue or organ-specific fashion). Exemplary genes that are generally inaccessible (with the exception of specific cell types) include, but are not limited to, hemoglobin-beta chain (HBB) and immunoglobulin light chain kappa (IGK).

In some embodiments, the DNA region is a gene sequence which has different accessibility depending on the disease state of the cell or otherwise have variable accessibility depending on type of cells or growth environment. For example, some genes are generally inaccessible in non-cancer cells but are accessible in cancer cells. Examples of genes with variable accessibility include, e.g., Glutathione-s-transferase pi (GSTP1).

In some embodiments, the DNA regions are selected at random, for example, to identify regions that have differential accessibility between different cell types, different conditions, normal vs. diseased cells, etc.

### VIII. Kits

The present invention also provides kits for performing the assays of the present invention. A kit can optionally include written instructions or electronic instructions (e.g., on a CD-ROM or DVD). Kits of the present invention can include, e.g., a first DNA endonuclease; a second DNA endonuclease different from the first DNA endonuclease; a first detectable label; a second detectable label different from the first detectable label; and in some embodiments, a DNA polymerase; and/or a ligase. In some embodiments, the first endonuclease is fused to a protein domain having affinity to a particular histone modification or cytosine methylation as detailed elsewhere herein. In some embodiments, the first endonuclease is a DNase. In some embodiments, the second endonuclease is a sequence-specific restriction endonuclease. In some embodiments, the first and second labels are fluorescent labels. In some embodiments, the labels are linked to deoxynucleotide triphosphates. In some embodiments, the kits further comprise a cell permeabilizing agent and/or formalin or formaldehyde. The kits can also comprise any of the components described herein with regard to the methods, including but not limited to, control DNA, appropriate buffers, etc.

### EXAMPLES

The following examples are offered to illustrate, but not to limit the claimed invention. The Example provides one possible method for performing the methods described herein.

A Permeabilization/DNAse treatment
1. Label one 1.5 ml tube “C” and another 1.5 ml tube “D” for “control” and “digestion” respectively. Into the “C” tube transfer 100 μl of chromatin buffer (10 mM Tris pH 7.4, 2.5 mM MgCl₂, 0.5 mM CaCl₂, and 0.25% w/v L-α-Lysophosphatidylcholine) for each control sample and an additional 50 μl of chromatin buffer. Transfer the same amount of chromatin buffer into the “D” tube. For example, if you have 6 control samples and 6 digestion samples to harvest, transfer 650 μl of chromatin buffer into tubes C and D. Warm tubes C and D to 37° C. for at least 5 minutes or until the digestion buffer (i.e., chromatin buffer plus enzyme) is clear.
   The second column below lists the amount of chromatin buffer to add to tubes C and D for commonly used numbers of samples.

| # of Samples | Chromatin Buffer | Nuclease (D tube only) |
|---|---|---|
| 3 | 350 μl | 7 μl |
| 6 | 650 μl | 13 μl |
| 9 | 950 μl | 19 μl |
| 12 | 1250 μl | 25 μl |

2. Add 2 μl of DNase I for every 100 μl of chromatin buffer in tube D. For example, if tube D contains 350 μl of chromatin buffer, add 7 μl of optimized nuclease. See third column in table above. Mix gently and store at room temperature.
3. Transfer suspended cells to a centrifuge tube (this step can be done in a round-bottomed 96-well plate when a large number of samples is being analyzed). Pellet cells (centrifuge at 200 g for 5 min) and remove supernatant.
4. Add 100 μl of chromatin buffer (no nuclease added) to each control tube.
5. Add 100 μl of digestion buffer to each digestion sample tube.
6. Resuspend cells by gently pipetting. Incubate at 37° C. for 1 hour.
7. Add 10 μl of 100 mM EDTA and proceed onto agarose embedding of the cells.

B Agarose Embedding

CHEF Genomic DNA Plugs Kit (Bio-Rad) are used. Embedding the cells in agarose preserves the DNA and avoids shearing that results in most DNA purification methods. Once embedded, the cells will be treated by Proteinase K and the DNA will be remain safe and exposed to subsequent enzymatic reactions in the agarose matrix.
1. Melt the 2% CleanCut agarose solution using a microwave and equilibrate the solution at 50° C. in a water bath
2. Pellet cells (centrifuge at 200 g for 5 min) and remove 80 of supernatant. Equilibrate the cell suspension at 50° C. for 2 minutes
3. Add 20 p. 1 of agarose solution (final concentration 0.8%), and mix gently at 50° C. Remove the tubes from water bath and allow the agarose to solidify at the bottom of the tube by placing tubes at 4° C. for 10-15 minutes.
4. Resuspend Proteinase K in Reaction Buffer (20 μl of enzyme stock per 1 ml of buffer). Add 200 μl to each tube and incubate at 50° C. for 2-16 hours
5. Wash the plugs in 1× Wash Buffer (1 ml per plug) for 30 minutes at room temperature. Remove Wash Buffer.
6. Repeat wash (step 5) with 1 ml 1× Wash Buffer+1 mM PMSF (in order to inactivate residual Proteinase activity) for 30 minutes at room temperature. Remove buffer.
7. Wash the plugs twice with 1 ml 1× Wash Buffer for 15 minutes at room temperature.
8. Proceed to the Nick translation step or store the plugs at 4° C. in 1× Wash Buffer. The plugs should be stable for up to 3 months.

C Fluorescent Labeling
1. Prepare 1×NT buffer (Composition of 10×NT buffer: 500 mM Tris, pH 7.5, 100 mM MgCl₂, 10 mM DTT, 0.5 mg/ml BSA)+10 mM beta-mercaptoethanol
2. Wash the plugs with 1 ml 1×NT+BME Buffer for 15 minutes at room temperature.
3. Remove the buffer and add 200 μl of labeling mix to each plug.

Composition of labeling mix:

1×NT buffer

10 mM beta-mercaptoethanol

50 uM each d(ACG)TP

33-43 um dTTP

7-17 um labeled dUTP (fluorescent label)

0.25 U E. Coli polymerase I/ul reaction

Water, to the desired final volume
4. Incubate at 15° C. for 2 hours
5. Wash twice the plugs with 1 ml 0.01% Triton X-100 for 30 minutes at 4° C. Proceed to ligation step.

D Ligation
1. Wash the plugs with 1 ml 1× Ligase buffer for 30 minutes at 4° C. (composition of 10× ligase buffer: 500 mM Tris-HCl, 100 mMMgCl₂, 10 mM ATP, 100 mM DTT, pH 7.5).
2. Remove the buffer and add 200 μl of ligation mix to each plug (1× ligase buffer+10 units of T4 Ligase/reaction)
3. Incubate at 15° C. for 2 hours
4. Wash twice the plugs with 1 ml 0.01% Triton X-100 for 30 minutes at 4° C. Proceed to second endonuclease step.

E Sequence-Specific Endonuclease Treatment
1. Wash the plugs with 1 ml 1× digestion buffer for 30 minutes at 4° C. (buffer dependent on the enzyme chosen)
2. Remove the buffer and add 200 μl of digestion mix to each plug (1× digestion buffer+10-100 units of nicking endonuclease/reaction)
3. Incubate at 15° C. for 2-16 hours
4. Wash twice the plugs with 1 ml 0.01% Triton X-100 for 30 minutes at 4° C. Proceed to fluorescent labeling step.
F Fluorescent Labeling
1. Prepare 1×NT buffer (Composition of 10×NT buffer: 500 mM Tris, pH 7.5, 100 mM MgCl₂, 10 mM DTT, 0.5 mg/ml BSA)+10 mM beta-mercaptoethanol
2. Wash the plugs with 1 ml 1×NT+BME Buffer for 15 minutes at room temperature.
3. Remove the buffer and add 200 μl of labeling mix to each plug.

Composition of labeling mix:

1×NT buffer

10 mM beta-mercaptoethanol

50 uM each d(ACG)TP

33-43 um dTTP

7-17 um labeled dUTP (fluorescent label different from 1^{st} label)

0.25 U E. Coli polymerase I/ul reaction

Water, to the desired final volume
4. Incubate at 15° C. for 2 hours
5. Wash twice the plugs with 1 ml 0.01% Triton X-100 for 30 minutes at 4° C. If experimental data suggest its necessity, a final ligation step could be done prior to analysis of the DNA (see Step D).

Depending on the platform for linearized DNA visualization, the samples may be used in the agarose plug configuration or may need to be in solution. If this is the case, apply the following protocol.

G Agarose Removal (Optional)
1. Remove the buffer and wash the plugs in 1×β-Agarase buffer (New England Biolabs) for 30 minutes at room temperature.
2. Remove the buffer and melt each agarose plug by incubation at 65° C. for 10 minutes.
3. Cool to 42° C. and incubate with 1 unit of β-Agarase at 42° C. for 1 hour

If a third nicking step is required for better mapping accuracy, steps D, E, and F should be repeated with a different sequence-specific nicking endonuclease and a labeling using a third type of fluorophore.

The pattern of restriction sites will be acquired in high resolution and analysed in comparison with virtual maps derived from the known genomic sequence.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of analyzing chromatin structure and/or epigenetic status in chromosomal DNA of a cell, the method comprising:
(i.) contacting a first DNA endonuclease to nuclear DNA in the cell, thereby introducing single-stranded nicks in the nuclear DNA;
(ii.) labeling the nicks in the DNA with a first detectable label to generate labeled nicks;
(iii.) ligating the labeled nicks in the DNA;
(iv.) purifying the DNA from the cell to generate purified genomic DNA;
(v.) after steps (i.)-(iv.), contacting a second DNA endonuclease to the purified genomic DNA to introduce single stranded nicks in the purified genomic DNA, wherein the second DNA endonuclease is different than the first endonuclease;
(vi.) labeling the nicks introduced by the second endonuclease in the purified genomic DNA with a second detectable label that is distinguishable from the first detectable label; and
(vii.) determining the identity of a region of interest in the genomic DNA based on the number and/or pattern of the second detectable labels in the genomic DNA and determining the presence, absence, or quantity of the first detectable labels in the region of interest, wherein the presence or quantity of the first detectable labels corresponds to chromatin structure and/or epigenetic status in the DNA region of interest in the nucleus of the cell.

2. The method of claim 1, further comprising, between steps (vi.) and (vii.), ligating the labeled nicks in the purified genomic DNA.

3. The method of claim 1, wherein steps (ii.)-(iii.) occur before step (iv.).

4. The method of claim 1, wherein steps (ii.)-(iii.) occur after step (iv.).

5. The method of claim 1, wherein prior to step (i) a cell membrane and nucleus of the cell is permeabilized to allow for entry of the first endonuclease into the nucleus.

6. The method of claim 1, wherein the first endonuclease is a DNase.

7. The method of claim 6, wherein the DNAse is DNase I.

8. The method of claim 1, wherein the second endonuclease is a sequence-specific single-stranded restriction endonuclease.

9. The method of claim 8, wherein the restriction endonuclease recognizes a 4, 5, 6, 7, or 8 base pair recognition sequence.

10. The method of claim 1, wherein the first and second detectable labels are fluorescent labels that produce signal at different wave lengths from each other.

11. The method of claim 1, wherein step (vii) comprises:
linearizing DNA molecules generated in step (vi) and recording the position of the first and/or second labels on the DNA molecules; and
correlating the position of the second labels on the DNA molecules to a region of interest in the genomic DNA.

12. The method of claim 11, wherein the linearizing step comprises passing single DNA molecules generated in step (vi) through a nanochannel.

13. The method of claim 12, wherein the passing step is performed in parallel with multiple single DNA molecules in a plurality of nanochannels.

14. The method of claim 1, wherein the accessibility of the first enzyme to the nuclear DNA is correlated with chromatin state.

15. The method of claim 1, wherein the first enzyme is linked to an affinity agent having affinity for a histone modification or cytosine methylation such that the accessibility of the first enzyme to the nuclear DNA is correlated with lack of histone modification status or methylation status.

16. The method of claim 15, wherein the affinity agent is an antibody.

17. The method of claim 15, wherein the affinity agent has affinity for a histone modification.

18. The method of claim 15, wherein the affinity agent has affinity for methylated DNA.

19. The method of claim 16, wherein the affinity agent is Methyl CpG binding protein (MBP).

20. The method of claim 15, wherein the histone modification is selected from histone methylation, histone acetylation, histone ubiquitinylation, histone phosphorylation, histone sumoylation, histone biotinylation, histone deimination, or DNA (e.g., adenosine or cytosine) methylation.

21. The method of claim 2, further comprising after step (vii) and before step (viii):
contacting a third DNA endonuclease to the purified genomic DNA to introduce single stranded nicks in the purified genomic DNA, wherein the third DNA endonuclease recognizes a different DNA recognition that the first and second DNA endonucleases; and
labeling the nicks in the purified genomic DNA with a third detectable label that is distinguishable from the first and second detectable labels.

22. The method of claim 1, wherein the method is performed on a single cell.

23. The method of claim 1, wherein the method is performed on a plurality of cells.

24. A kit, comprising
a first DNA endonuclease;
a second DNA endonuclease different from the first DNA endonuclease;
a first detectable label;
a second detectable label different from the first detectable label;
a DNA polymerase; and
a ligase.

25. The kit of claim 24, wherein the first endonuclease is fused to a protein domain having affinity to a particular histone modification or cytosine methylation.

26. The kit of claim 24, wherein the first endonuclease is a DNase.

27. The kit of claim 24, wherein the second endonuclease is a sequence-specific restriction endonuclease.

28. The kit of claim 24, wherein the first and second labels are fluorescent labels.

29. The kit of claim 28, wherein the labels are linked to deoxynucleotide triphosphates.

30. The kit of claim 24, further comprising a cell permeabilizing agent.
